(19) United States
(12) Patent Application Publication
(10) Pub. No.: US 2012/0055473 A1
(43) Pub. Date: Mar. 8, 2012

(54) APPARATUS, SYSTEM AND METHOD FOR BRONCHIAL THERMOPLASTY

(76) Inventor: Noam Gavriely, Haifa (IL)

(21) Appl. No.: 13/255,908

(22) PCT Filed: Mar. 10, 2010
(86) PCT No.: PCT/IL10/00201

§ 371 Date: Nov. 21, 2011

Related U.S. Application Data
(60) Provisional application No. 61/158,752, filed on Mar. 10, 2009.

(51) Int. Cl. A61M 16/10

(52) U.S. Cl. 128/204.17

(57) ABSTRACT

The present invention relates to an apparatus, system and a method for producing heat shock in a targeted area.

### FIELD OF THE INVENTION

The present invention relates to an apparatus, system and a method for producing heat shock in a targeted area.

### BACKGROUND OF THE INVENTION

Asthma is a disease of the bronchial airways of the lung. Generally, asthma is caused by reversible intermittent contraction of the smooth muscles in the airway walls, swelling or edema of the airway wall and increased secretions into the airway lumen. The airways become constricted leading to increased resistance in the airways that in turn causes a patient to labor harder in order to get air in and out of the lung.

The central airways comprising bronchi from generations 0-6 have been identified as primary section of the lung where airway resistance is present at heightened levels. Accordingly, research surrounding that section of the lung has lead to the thermoplasty treatments. Recently research has revealed that heating the airways to high temperatures, for example 70 degree Celsius, for a specific duration of time is an effective treatment of asthma. It is believed that thermoplasty applied to a bronchial section by inserting a heat delivery catheter stops the smooth muscle of the bronchi in the treated segment from contracting and from narrowing of the airway lumen. An example of such applications is taught in U.S. Pat. Nos. 7,200,445; 7,027,869; 7198635; 7104987 commonly assigned to Asthmatx, Inc which teaches the use of a delivery catheter to deliver heat to a localized bronchial section of the lungs.

Typically, the treatment consists of 3 procedures performed under general anesthesia during which multiple airway locations are heated. Clinical studies show that while such treatment is effective, it is only effective in the short term lasting for about 1-2 years [1].

### SUMMARY OF THE INVENTION

There is an unmet need for, and it would be highly useful to have, an apparatus, system and a method for improved minimally invasive bronchial thermoplasty procedures that may preferably be performed on an outpatient basis, may be performed without general anesthesia and may therefore be performed at a higher frequency on an as needed basis.

It is an object of the present invention to overcome the deficiencies of the current bronchial thermoplasty treatment by providing a minimally invasive apparatus, system and method for bronchial thermoplasty treatment to patients suffering from asthma.

Within the context of this invention the term ‘heat shock” may be used interchangeably with the term “thermal energy” to indicate the delivery of energy most preferably in the form of thermal energy that is optionally and preferably delivered in a gas-phase bolus form.

A preferable embodiment of the present invention provides for an apparatus that preferably induces heat shock in a bronchial region, optionally extending from the trachea (zero generation) up to the sixth generation bronchi, more preferably comprising first to sixth generation bronchi, more preferably second to sixth generation bronchi and optionally second to fifth generation bronchi. Optionally, bronchial heat shock according to the present invention is induced by targeted delivery of at least one or more heat carrying agents targeted to the desired bronchial region. More preferably, heat shock is induced by targeted delivery of at least two or more agents to the desired bronchial region. Most preferably, heat shock is induced by delivery of a plurality of agents to the desired bronchial region.

Targeted heat shock according to the present invention is optionally and preferably induced by a targeted delivery of a bolus of heated gas. Optionally heat shock is induced by the delivery of heated aerosolized agents. Optionally, heat shock is induced using a plurality of agent delivered in sequence so that a bolus delivery of at least one agent will cause an exothermic reaction in the targeted region. For example, a first agent may be delivered to a targeted area and activated with the bolus delivery of a second agent preferably the combination of which induces an exothermic reaction between the first and second agents in the targeted region. Optionally a third agent may be provided to neutralize or otherwise terminate the delivery of heat shock.

Optionally, at least one or more heat shock agents may be applied in a graded manner preferably producing a heat shock gradient within the chosen bronchial region.

Optionally, at least one or more heat shock agents are delivered sequentially in individualized bolus dosage form or in a graded single bolus form. Optionally each agent may be delivered to a different respiratory region. For example a first agent may be delivered to the alveolar region while a second agent to the bronchial region and a third agent to the upper respiratory tract.

Optionally, at least one or more heat shock agents are delivered sequentially in individualized bolus dosage form.

Optionally, at least one or more heat shock agents may be delivered in a multi bolus dosage form.

Optionally, at least one or more heat shock agents are delivered with successive breathing cycles, wherein delivery may be passive or assisted.

Optionally, at least one or more heat shock agents may be delivered in a targeted manner, most preferably a first agent targeted to the bronchial region of interest that is optionally and preferably activated to produce heat shock when reacted with the delivery of a second agent. For example, a first agent may take the form of Calcium Oxide while a second agent may be provided in the form of water that preferably activates the heat shock reaction.

Optionally heat shock reaction may be in the form of an oxidation reduction reaction between a first agent and a second agent. Optionally, a first agent may be an oxidant while second agent is a reducing agent, or optionally a first agent is a reducing agent while the second agent is an oxidant reacted to bring about the required heat shock reaction. For example an oxidant may take the form of potassium chlorate, potassium perchlorate, potassium dichromate, cesium nitrate, and potassium nitrate; while a second reducing agent may take the form of various organic materials for example including but not limited to rubber, polymeric materials, epoxy resin, phenol formaldehyde resin, and the like. For example, the exothermic reaction may optionally feature one or more of a polymerization reaction, any type of redox reaction (for example featuring an oxidant, such as potassium perchlorate, potassium dichromate, potassium nitrate, potassium chlorate, cesium nitrate, or the like, with a second reactant that is preferably capable of acting as a reducing agent which may be one or more of various organic materials, such as rubber, polymeric materials, epoxy resin, phenol formaldehyde resin, and the like, or which may be phosphorus, sulfur, and the like), the reaction between sulfuric acid and sugar, or a reaction which features potassium chlorate, potassium perchlorate, potassium dichromate, cesium nitrate, and/or potassium nitrate, with a second reducing agent (which may be an organic agent as described above or any other type of agent), or any type of reaction involving oxygen (for example, see PCT Publication No. WO/1993/017961, hereby incorporated by reference as if fully set forth herein, or any reaction between metal and oxygen, for example with powdered iron).

Optionally, heat shock agents for example may include but are not limited to thermal energy, exothermic agent, endothermic agent, hot gas, gas with special heat conductivity properties, Helium, heating element, heated aerosol of liquid, heated aerosol of particles, powder, controllable exothermic agent, reactive exothermic agent, reactive endothermic agent., radioactive gas, radioactive aerosol, emitting alpha rays, beta rays or gamma rays, electrically charged ions, heated powder, fine particles, heated fine particles, biologically active material.

Optionally, heat shock is induced by heating the bolus of gas or gas-containing elements with high heat capacity or conductivity. Such heating can be in a separate compartment or by temporarily activating reacting elements in the air stream in a mouthpiece or near the tip of an endotracheal tube. Optionally, such heating is synchronized with the respiratory cycle.

Optionally, heating is provided by transiently activating a heating element that is incorporated with a user interface to bring about heat. A heating element may optionally be embedded in an endotracheal tube, face mask or the like optionally to provide a heat source to at least one or more heat shock agent.

Optionally heat shock is induced by a reaction of at least two or more heat shock agents in the targeted bronchial region. Optionally, heat shock is induced by direct delivery of one heat shock agent in the targeted bronchial region. Optionally heat shock is induced by a plurality of graded agents sequentially delivered to the targeted bronchial region.

Optionally, a first bolus coats the targeted airways with a first reagent, and then the first agent is activated with a second agent to initiate an exothermic reaction. Optionally, control of the amount of heat generated may be provided by using a cooling agent to stop the reaction.

Optionally, the heat shock agent may be stored to be delivered from at least one or more reservoir.

Optionally the entire process may be preceded by administration of a bronchodilating agent such as albuterol inhalation to assure that all airways are optimally open.

Optionally, the heat sock agent may be optionally delivered using at least one user interface for example including but not limited to an endotracheal tube (‘ETT’), mask, a facemask, thermally insulated ETT, bronchial blocker, double lumen tube, bronchoscope, or the like. Optionally, heating is provided by a heating element that is incorporated with a user interface to bring about heat. A heating element may optionally be embedded in an endotracheal tube , face mask or the like optionally to provide a heat source to at least one or more heat shock agent.

Optionally, heat shock agents delivery may be facilitated by at least one or more means for example including but not limited to spontaneous breathing, artificial positive pressure ventilation, high frequency, low volume oscillatory ventilation, jet ventilation or the like.

An optional embodiment of the present invention provides for an apparatus for delivering heat shock to a bronchial region comprising at least one or more heat shock agent compartment comprising at least one or more heat shock agents and connected to a user interface by piping; and wherein the piping comprises at least one or more controllable valves to control circulation of a flowing fluid between the heat shock agent compartment and the user interface; and wherein the controllable valves are controlled with a controller.

Optionally the apparatus according may further comprise a heat shock agent dispenser for example including but not limited to a pump, ventilator or the like. Optionally the heat shock agent dispenser may be disposed within a heat shock agent compartment. Optionally the dispenser may be provided to dispense the at least one or more heat shock agent within the flowing fluid.

Optionally a user interface provided with an optional embodiment according to the present invention may further comprises a sensor, for example including but not limited to flow sensor, volume sensor, pressure sensor, temperature sensor or the like.

Optionally the apparatus according to the present invention may be provided with controller function that may be determined according to an assessment of the pulmonary tract. Optionally the pulmonary tract assessment and/or analysis may be facilitated by means for example including but not limited to software tools, imaging devices, physiological measurements, physiological measurements of the anatomical dead-space, X-ray, Computerized Tomography (CT), Magnetic Resonance Imaging (MRI) , Positron Emission Tomography (PET), ultrasound, nuclear imaging, radioactive gases and any combination thereof.

Optionally the apparatus according to an optional embodiment of the present invention may further comprise a controllable transitional compartment for directing a flowing fluid to a user interface. Optionally the transitional compartment may comprise at least one or more holding compartment for sorting the heat sock agents for delivery.

Optionally the apparatus according to an optional embodiment of the present wherein at least one valve is provided in the form of an exhalation valve, optionally provided to control the breathing cycle and delivery of the at least one heat shock agent.

An optional embodiment of the present invention provides for the apparatus according to an optional embodiment of the present that may further comprise a respiratory tract assessor for assessing the respiratory tract prior optionally and preferably for determining the heat shock agents to deliver and parameters associated thereto.

An optional embodiment of the present invention provides for a method for delivering heat shock to a bronchial region using the apparatus according to optional embodiments of the present invention the method comprising; assessing the pulmonary tract; and determining parameters of at least one or more heat shock agents; and preparing relative dosages of the heat shock agents; and delivering the heat shock agents to the bronchial region. Optionally the method according to the present invention may further comprise a pulmonary tract assessment following heat shock agent delivery; and a neutralizing the heat shock agents.

Optionally the heat shock agents may be delivered in a controllable manner for example including but not limited to sequential delivery, intermittent delivery, or continuous delivery. Optionally sequential delivery comprises sequential delivery of a plurality of heat shock agents for example including but not limited to a insulating agent, a heat shock priming agent, at least one or more heat shock generating agent, and a heat shock neutralizing agent.

Optionally the pulmonary tract assessment determines the distribution heat shock agents to different targeted bronchial regions. Optionally each of agents may be delivered to targeted bronchial regions within the pulmonary tract.

Optionally at least two or more heat shock agents may be utilized to produce heat shock to a targeted bronchial region within the pulmonary tract.

Optionally the pulmonary tract assessment and/or analysis may be facilitated by means for example including but not limited to software tools, imaging devices, physiological measurements, physiological measurements of the anatomical dead-space, X-ray, Computerized Tomography (CT), Magnetic Resonance Imaging (MRI) , Positron Emission Tomography (PET), ultrasound, nuclear imaging, radioactive gases and any combination thereof.

Unless otherwise defined, all technical and scientific terms used herein have the same meaning as commonly understood by one of ordinary skill in the art to which this invention belongs. The materials, methods, and examples provided herein are illustrative only and not intended to be limiting. Implementation of the method and system of the present invention involves performing or completing certain selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic block diagram of an optional apparatus according to the present invention;

FIGS. 2A-B are schematic block diagrams of an optional apparatus according to the present invention;

FIG. 3 is an exemplary flowchart of a method according to the present invention.

### DESCRIPTION OF THE PREFERRED EMBODIMENTS

The present invention is of an apparatus, system and a method for producing heat sock in a targeted bronchial region of the respiratory tract. An optional embodiment of the present invention provides for the delivery of a single bolus or multiple boli in succession of an agent to a target within the bifurcating network of the pulmonary airways. Optionally, the agent includes but is not limited to matter or energy capable of selectively reaching the airway walls and interact with them, most preferably to produce heat shock. Optionally, the heat shock agent may be delivered in various states, for example including but not limited to, aerosolized liquids or gas, to the desired portion of the respiratory tract by controlling the delivery parameters of the agents. For example, the agent's delivery parameters may optionally include but is not limited to state, timing, concentration, order, volume, pressure or the like. Preferably, control of the delivery parameters provide the ability to determine the location where at least one or more agent is to be delivered.

The principles and operation of the present invention may be better understood with reference to the drawings and the accompanying description.

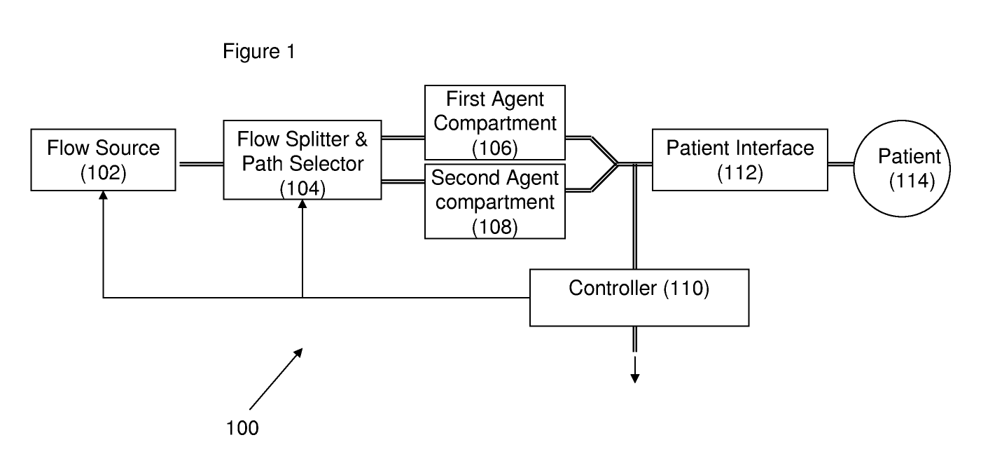

Referring now to the drawings, FIG. 1 is a schematic block diagram of an exemplary apparatus according to the present invention for device that induces heat shock within a targeted bronchial region of the respiratory system. FIG. 1 shows an apparatus 100 according to an optional embodiment of the present invention comprising a flow source 102, flow splitter 104, a first agent compartment 106, a second agent compartment 108, respiratory controller 110, and a patient interface 112.

Most preferably patient interface 112, for example including but not limited to a mask, facemask, ETT, thermally insulated ETT, bronchial blocker, double lumen tube or the like, provides a means to deliver at least one or more agents to a patient 114 specifically targeted to a bronchial region. Preferably patient interface 112 comprises a sensor (not shown) for example including but not limited to a flow sensor, volume sensor, pressure sensor, temperature sensor. Most preferably, the sensor (not shown) may be attached, incorporated, integrated or otherwise coupled to patient interface 112. Optionally and preferably the targeted bronchial region is up to a sixth generation bronchi.

Most preferably flow source 102 provides a fluid medium through which at least one or more agent is delivered to the targeted bronchial region. For example, flow source 102 provides the fluid flow required to reach the targeted region providing sufficient pressure, volume, rate as required. Optionally, flow source 102 includes but is not limited to a spring loaded sac, a pump, ventilator, nebulizer, solenoid controlled pressurized gas reservoir or the like. Optionally, flow source 102 is able to control at least one or more parameters for example including but not limited to fluid volume, fluid pressure, fluid flow rate, delivery timing, timing with inspiration or the like parameters.

Optionally, flow source 102 provides fluid medium for example including but not limited to air, pressurized air, gas or the like as is known and accepted in the art, to a flow splitter 104. Preferably flow splitter 104 channels a controllable amount of fluid medium to at least one or more compartments comprising heat shock agents. For example, flow splitter 104 provides the required fluid flow to a first heat shock agent compartment 106 and to a second heat shock agent compartment 108. According to an optional embodiment of the present invention a plurality of heat shock agents and compartments may be disposed within apparatus 100. Optionally, flow splitter 104 may for example be provided in the form including but not limited to a valve, a controllable valve, a switch, or the like as known in the art. Heat shock agent compartments 108 and 106 may optionally comprise a nebulizer or similar means to load a flowing fluid with at least one or more heat shock agents. Optionally, fluid flow from a first agent compartment 106 to patient interface 112 may be mediated by a controllable valve (not shown). Optionally, fluid flow from a second agent compartment 108 to patient interface 112 may be mediated by a controllable valve (not shown). Optionally, fluid flow from compartments 108 or 106 may be controllable providing controlled timing, rate, volume or pressure.

Preferably, fluid flow from the various components comprising apparatus 100 may be controllable with controller 110 providing overall inhalation and exhalation control with patient 114 through interface 112. Optionally, controller 110 facilitates the control of a plurality of controllable parameters for example including but not limited to overall fluid flow, fluid pressure, fluid volume, fluid rate, fluid timing, valves, switches or the like.

Optionally, heating may be provided by a heating element (not shown) and may optionally incorporated with user interface 112 to bring about heat of agent. A heating element may optionally be embedded at the distal end of an endotracheal tube, face mask or the like optionally to provide a heat source to at least one or more heat shock agent.

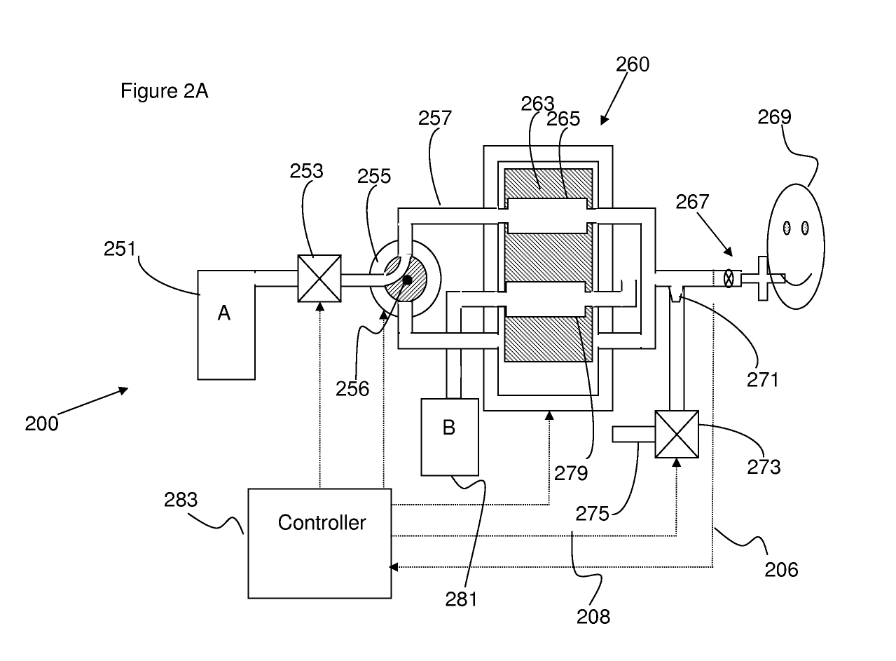

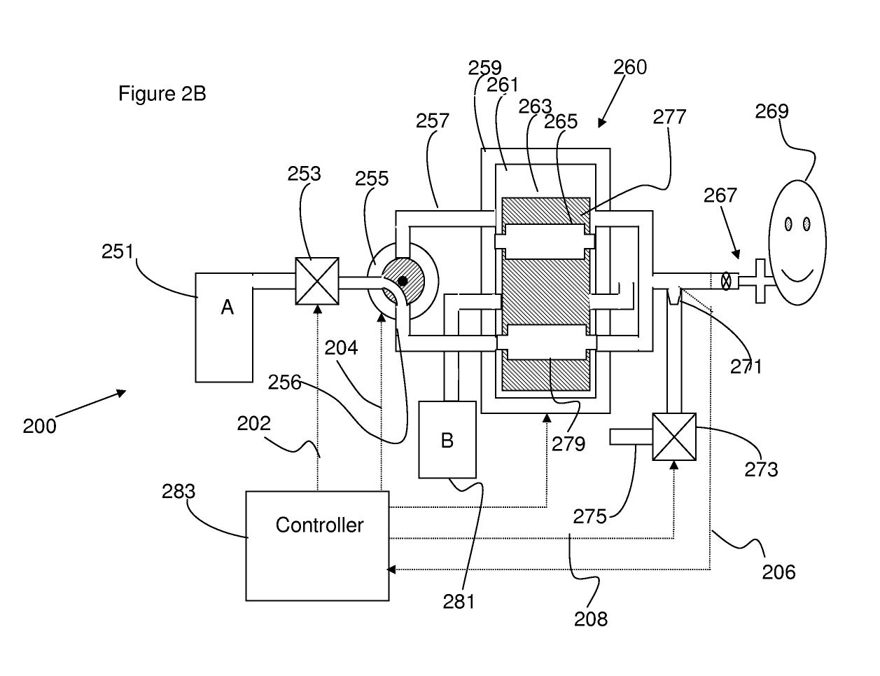

FIGS. 2A-B are a schematic block diagrams of an optional apparatus 200 according to the present invention depicting two optional states of apparatus 200. Apparatus 200 preferably comprises controller 283, at least one or more heat shock agent compartments, for example a first heat shock agent compartment 251 and a second heat shock agent compartment 281. FIGS. 2A and 2B depict two states of apparatus 200 wherein preferably at least one heat shock agent preferably stored in a first compartment 251 or a second compartment 281 may be delivered to a patient 269 by piping a flowing fluid through a transitional compartment 260 to a user interface 267. Optionally piping a flowing fluid through apparatus 200 may optionally be accomplished with spontaneous breathing, partially assisted breathing, or ventilated breathing. Most preferably, all components of apparatus 200 are controlled by a controller 283. Controller 283 preferably, controls the activity of transitional compartment 260 and a plurality of valves for example including but not limited to valves 253, 255 and 273.

Optionally, user interface 267 may for example be provided in the form including but not limited to an endotracheal tube (ETT), mask, a facemask, thermally insulated ETT, bronchial blocker, double lumen tube, bronchoscope, or the like. Optionally, heating may be provided by a heating element that may be incorporated with a user interface to optionally induce heating of at least one heat shock agent. A heating element may optionally be embedded in an endotracheal tube, face mask or the like optionally to provide a heat source to at least one or more heat shock agent.

Preferably, transitional compartment 260 comprises at least one, more preferably two and most preferably a plurality of holding compartments for example holding compartments 279 and 265. Optionally holding compartments 279 or 265 may comprise at least one or more controllable elements (not shown) for example including but not limited to heating element, sensors, spring, valve, solenoid, or the like. Optionally, the dimensions, shape, characteristics and parameters associated with holding compartments 279 and 265 are controllable and may change in accordance to at least one or more parameters of an associated heat shock agent. Preferably transitional compartment 260 comprises a controllably moveable platform 263 that may be preferably controllable with controller 283. Most preferably platform 263 may be automatically controlled in accordance with the respiratory cycle of patient 269, optionally platform 263 may be manually controlled by a user. Optionally, moveable platform 263 provides a user with the option of selecting which holding compartment 279 or 265 may be directly linked to user 269 via piping to allow a flowing fluid to pass therethrough.

FIG. 2A depicts the flow of a heat shock agent from a first compartment 251 through to holding compartment 265 reaching patient 269 while second compartment 281 delivers a second heat shock agent to holding compartment 279, most preferably at atmospheric pressure or near atmospheric pressure or optionally under pressure. FIG. 2B provides a depiction of the flow of a heat shock agent from holding compartment 279 to patient 269.

Apparatus 200 provides heat shock to a bronchial region (not shown) of patient 269 preferably through user interface 267 and exhalation opening 271. Optionally, first heat shock agent compartment 251 comprises a heat shock agent for example including but not limited to air, oxygen, helium, heliox, endothermic agent, or the like agent. Optionally and preferably first heat shock agent targeted to the lower bronchial regions, for example including but not limited to the alveolar regions, wherein heat shock is not wanted preferably, first agent originating in compartment 251 therefore provides protection or insulation from heat shock that may be applied to upper portions of the respiratory tract, for example including but not limited to up to sixth generation bronchi, optionally extending from the trachea (zero generation) up to the sixth generation bronchi, more preferably first to sixth generation bronchi, more preferably second to sixth generation bronchi and optionally second to fifth generation bronchi

Preferably, the fluid flow of a first heat shock agent leaving compartment 251 may be controllable with a valve 253. Preferably, valve 253 may be controllable with controller 283, that preferably governs valve control through at least one or more parameters for example including but not limited to fluid flow rate, fluid volume, fluid temperature, fluid pressure, or the like. Optionally and preferably, the direction of flow of a first fluid heat shock agent may be controlled by controller 283 through valve 255 for example including but not limited to a rotational valve, solenoid valve, or the like. Optionally, valve 255 preferably comprises at least one or more pivotal joint 256 to depict and control the direction of fluid flow to transitional compartment 260. preferably leading to user interface 267. Most preferably, while a first heat shock agent from compartment 251 controllably flows through to transitional compartment 260 through holding compartment 265 to user interface 267 to patient 269; a second heat shock agent preferably flows to holding compartment 279 from a second heat shock compartment 281, as depicted in FIG. 2A. Optionally, fluid flow from compartment 251 to patient 269 is optionally mediated by artificial breathing or spontaneous breathing.

Optionally, a second heat shock agent targeting a different pulmonary region optionally and preferably up to sixth generation bronchi, is optionally utilized to induce heat shock in the targeted area. FIG. 2B, depicts the delivery of a second heat shock agent, preferably originating from second heat shock agent compartment 281 and stored in holding compartment 279 to patient 269 through user interface 267, most preferably at about atmospheric pressure. Optionally, second heat shock agent flowing through user interface 267 may be heated through an imbedded or otherwise associated heating element comprising the user interface.

Optionally and preferably, the fluid delivery of at least one or more heat shock agent to a patient 269 through piping of apparatus 200 may be further controlled by a controller controlled exhalation valve 273. Preferably, exhalation valve controls the opening and closing of exhalation opening 271 to optionally control the breathing cycle of a patient 269 in delivering at least one or more preferably a plurality of heat shock agents to a zero to sixth generation bronchi.

The transition from apparatus depicted in FIG. 2A to apparatus 200 depicted in FIG. 2B is optionally dependent on the optional positioning of moveable platform 263, preferably controllable with controller 283. Optionally, movement of moveable platform 263 may be dependent on the automatic breathing cycle of patient 269 that may be optionally ventilated, spontaneous or partially spontaneous

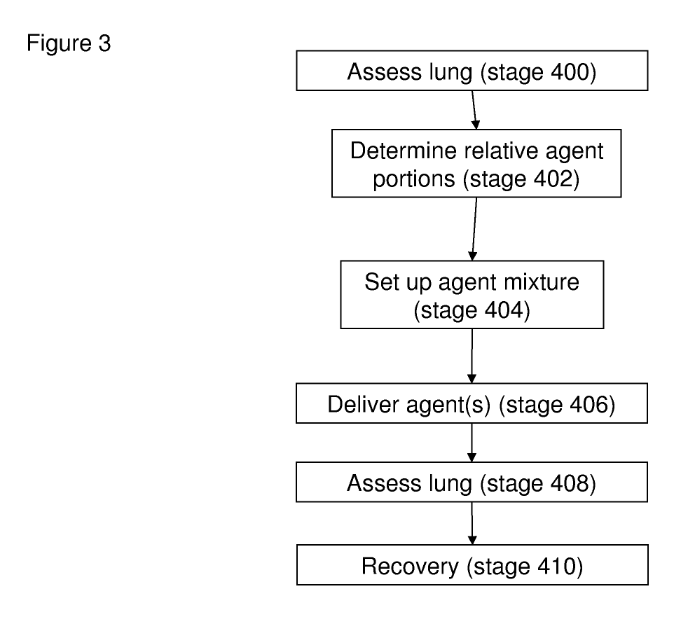

FIG. 3 depicts a method of use of the apparatus according to an optional embodiment of the present invention to preferably induce heat shock within a predetermined bronchial region. In stage 400 the lungs of a user may be assessed optionally to determine the lung capacity, the fluid volume required to reach specific locations within the targeted bronchial regions. For example, if targeting first to fourth bronchial regions its corresponding fluid volume may be assessed and may later be used for example to control the heat shock agent parameters for example including but not limited to volume, rate, pressure, type of agent, timing or the like. Optionally, assessment and analysis may be provided by at least one or more, or a combination of software tools and/or imaging tools and/or physiological measurements of the anatomical dead-space. Imaging tools for example including but not limited to imaging of the lung by x-ray or CT or MRI or by nuclear imaging methods such as by using radioactive gases or a combination thereof.

In stage 402 based on the analysis process of stage 400 the heat shock agent parameters are determined for example including but not limited to volume, rate, pressure, timing, type of agent or the like. In stage 404 the determined parameters are preferably incorporated into the controller 283 as shown in FIG. 2, providing overall control to apparatus 200 of FIG. 2. In stage 406 an optional apparatus according to the present invention, for example as depicted in FIG. 2, may be used to provide heat shock optionally with at least one or more heat shock agents, that are delivered to a predetermined bronchial region according to the present invention. Optionally, an agent may be used to prime the targeted bronchial region so that a reaction between the agent and a heat shock agent produce the desired heat shock in the desired region. Optionally, a first heat shock agent may be delivered to insulate or protect the non targeted regions. Optionally, a plurality of agents are used to create selected regions and heat shock levels within the bronchial region. Following heat shock delivery of stage 406 the lungs are preferably reassessed in stage 408 to determine the efficacy of the treatment used. Optionally the treatment may be altered, continued or stopped. In stage 410 a recovery stage may be optionally utilized to clear the respiratory tract of any agents optionally remaining therein. Optionally, stage 410 may be implemented based on the assessment results of stage 408.

For example, the alveoli are not to receive heat shock while heat shock is required for second to fifth generation bronchi. Accordingly, the relative lung volume of each bronchi region may be assessed. A first agent, for example including but not limited to Helium-Oxygen mixture, is selected, its volume, pressure, timing and delivery rate may be determined in stage 400 and stage 402 to insulate or protect the lower pulmonary tract comprising the alveoli and up to sixth generation bronchi, from heat shock. A second agent may be selected to induce and produce heat shock, for example, in second to fifth generation bronchi, according to the corresponding fluid volume associated with second to fifth generation bronchi. Optionally, a third heat shock agent may be provided to insulate or protected the upper respiratory tract and up to the second generation bronchi. Optionally, a fourth agent may be used to remove or otherwise neutralize heat shock agents within the respiratory tract. During the delivery as depicted in stage 406, the first agent may be delivered followed by the second agent and finally with the third agent, optionally the third agent may be the same as the first however the required volume may be different. Preferably, each agent is geared toward a different and specific bronchial region. Optionally, delivery may be sequential or simultaneous. Optionally, a respiratory tract medicament may be provided to a user to allow a two stage reaction to produce the heat shock. For example, a drug may be given that specifically reacts with heat shock agent two to bring about bronchial thermoplasty once the drug interact with the required agent.

Optionally, a first agent may be utilized to target the tracheal and alveolar regions of the respiratory tract, preferably to provide heat shock protection or insulation. Optionally, a second agent may be provided to the targeted bronchial regions, for example first to sixth generation bronchi. Preferably, the first and second agents effectively line or coat the respiratory tract providing both heat shock protected as well as heat shock targeted area. Optionally with the exposure of a third agent that preferably selectively reacts with the second agent exothermically producing heat shock in the targeted area while not reacting with the first agent or optionally reacting endothermically with the first agent. Optionally, a fourth agent may then provided to preferably deactivate all agents within the respiratory tract.

### REFERENCES

1. R. H. Brown, W. Wizeman, C. Danek, and W. Mitzner, “In vivo evaluation of the effectiveness of bronchial thermoplasty with computed tomography.” J. Applied Physiology, 2005, vol. 98, pp. 1603-1606,

While the invention has been described with respect to a limited number of embodiments, it will be appreciated that many variations, modifications and other applications of the invention may be made.

What is claimed is:

1) An apparatus for delivering heat shock to a bronchial region comprising:
a) at least one or more heat shock agent compartment comprising at least one or more heat shock agents and connected to a user interface by piping; and wherein
b) said piping comprises at least one or more controllable valves to control circulation of a flowing fluid between said heat shock agent compartment and said user interface; and wherein said controllable valves are controlled with a controller.

2) The apparatus of claim 1 further comprising a heat shock agent dispenser.

3) The apparatus of claim 2 wherein said heat shock agent dispenser is disposed within said heat shock agent compartment.

4) The dispenser of claim 2 chosen from the group consisting of a pump and ventilator.

5) The dispenser of claim 2 wherein said dispenser is provided to dispense said at least one or more heat shock agent within said flowing fluid.

6) The apparatus of claim 1 wherein said user interface further comprises a sensor.

7) The user interface of claim 6 wherein said sensor is chosen from the group consisting of a flow sensor, volume sensor, pressure sensor, and temperature sensor.

8) The apparatus of claim 1 further comprising a controllable transitional compartment for directing said flowing fluid to said user interface.

9) The transitional compartment of claim 8 comprising at least one holding compartments for sorting said heat sock agents.

10) The apparatus of claim 1 wherein said at least one valve is an exhalation valve to control the breathing cycle and delivery of said at least one heat shock agent.

11) The apparatus of claim 1 wherein the function of said controller is determined according to an assessment of the pulmonary tract.

12) A system for delivering heat shock to a bronchial region comprising: an apparatus according to claim 1 and further comprising a respiratory tract assessor.

13) A method for delivering heat shock to a bronchial region using the apparatus of claim 1 comprising:
a) assessing the pulmonary tract; and
b) determining parameters of at least one or more heat shock agents; and
c) preparing relative dosages of said heat shock agents; and
d) delivering said heat shock agents to said bronchial region.

14) The method of claim 13 further comprising
e) pulmonary tract assessment following heat shock agent delivery; and
f) neutralizing said heat shock agents.

15) The method of claim 13 wherein said heat shock agents may be delivered in a controllable manner chosen from the group consisting of sequential delivery, intermittent delivery, or continuous delivery.

16) The method of claim 13 wherein said pulmonary tract assessment determines the distribution heat shock agents to different targeted bronchial regions.

17) The method of claim 15 wherein sequential delivery comprises sequential delivery of heat shock insulating agent, heat shock priming agent, at least one or more heat shock generating agent, and a heat shock neutralizing agent.

18) The method of claim 17 wherein each of said agents are delivered to targeted bronchial regions within the pulmonary tract.

19) The method of claim 13 wherein at least two or more heat shock agents are utilized to produce heat shock to a targeted bronchial region within the pulmonary tract.

20) The method of claim 13 wherein said pulmonary tract assessment analysis is facilitated by means selected from the group consisting software tools, imaging devices, physiological measurements, physiological measurements of the anatomical dead-space, X-ray, computer tomography (CT), magnetic resonance imaging (MRI), positron emission tomography (PET), ultrasound, nuclear imaging, radioactive gases and any combination thereof.
